(19) United States
(12) Patent Application Publication
(10) Pub. No.: US 2010/0285186 A1
(43) Pub. Date: Nov. 11, 2010

(54) Process for the Manufacture of Sugar and Other Food Products

(75) Inventors: David Kannar, Belgrave South (AU); Barry James Kitchen, Bon Beach (AU); Michael O'Shea, Runcorn (AU)

Correspondence Address: THE WEBB LAW FIRM, P.C., 700 KOPPERS BUILDING, 436 SEVENTH AVENUE, PITTSBURGH, PA, 15219, US

(73) Assignee: HORIZON SCIENCE PTY LTD, Braeside (AU)

(21) Appl. No.: 12/681,221

(22) PCT Filed: Oct. 2, 2008
(86) PCT No.: PCT/AU08/01461

§ 371 Date: Jul. 22, 2010

(30) Foreign Application Priority Data
Oct. 4, 2007 (AU) 2007905439
Aug. 7, 2008 (AU) 2008904050

(51) Int. Cl. G01N 33/02

(52) U.S. Cl. 426/231

(57) ABSTRACT

A process for manufacturing of sugar products having desired levels of specific phytochemicals comprising the steps of: preparing a primary sugar product; analysing said primary sugar product for its phytochemical profile using an analytical method selected from the group consisting of near infrared spectroscopy, electrical conductivity, and combinations thereof; comparing said profile; treating said primary sugar product, if required, to achieve a final sugar product having desired levels of specific phytochemicals.

### FIELD OF THE INVENTION

The invention relates to a process for the manufacture of food products, including low glycaemic index sugar products and other food products having desired phytochemical levels. In particular, a process for the manufacture of sugar products using near infrared spectroscopy or electrical conductivity to ascertain whether a sugar product has the desired profile of desirable phytochemical species, such as polyphenols, antioxidants, organic acids, colorants, polysaccharides, minerals, reducing sugars, policosanols, phytosterols, neutral lipids, phospholipids, emulsifiers, proteins and other phytochemicals.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

While the present invention is described with reference to profiles of polyphenols, antioxidants, organic acids, colorants, polysaccharides, minerals, reducing sugars, policosanols, phytosterols, neutral lipids, phospholipids, emulsifiers, protein for extracts of cane sugar or processing streams associated with cane sugar processing, the invention is not so limited but includes beet sugar extracts and beet sugar processing streams.

### Near Infrared Spectroscopy (NIR)

Spectroscopy is the science of identifying chemical compounds on the basis of their effect on specific wavelengths of electromagnetic radiation. Near infrared spectroscopy (NIR) is based on the analysis of absorption of radiation having a wavelength in the near infrared portion of the electromagnetic spectrum that is, from 400 to 2,500 nm.

### Electrical Conductivity (EC)

Electrical conductivity (EC) estimates the amount of total dissolved salts (TDS), or the total amount of dissolved ions in solution. EC is measured in microSiemens per centimeter (μS/cm) and is recorded using a sensor which consists of two metal electrodes.

### Cane Sugar Refining

After being mechanically harvested, sugar cane is transported to a mill and crushed between serrated rollers. The crushed sugar cane is then pressed to extract the raw sugar juice, while the bagasse (leftover fibrous material) is used for fuel. The raw juice is then heated to its boiling point to extract any impurities, then lime and bleaching agents are added and mill mud is removed. The raw juice is further heated under vacuum to concentrate and increase Brix value. The concentrated syrup is seeded to produce bulk sugar crystals and a thick syrup known as molasses. The two are separated by a centrifuge and the molasses waste stream is collected for use as a low-grade animal feedstock. A flow chart of this process appears below.

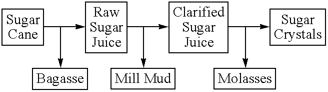

The sugar refining process generates a large number of products including raw juice, bagasse, mill mud, clarified juice and so forth.

The bulk sugar crystals from the above process are further refined to produce many commercially available sugar products. For example, the further refining may include mixing the bulk sugar crystals with a hot concentrated syrup to soften the outer coating on the crystals. The crystals are then recovered by centrifuge and subsequently dissolved in hot water. This sugar liquor is then further purified by carbonation or phosphotation, filtration, decolourisation and then seeded with fine sugar crystals. Once the crystals have grown to the requisite size, the crystals are separated from the syrup by (centrifugation), centrifuge, then dried, graded and packaged. There may be several repetitions of recovering sugar crystals from the sugar liquor. The dark sugar syrup which is left after all of the sugar crystals have been recovered is also called molasses.

### Sugar Composition

The composition of cane sugar products and waste streams are complex and quite variable—the chemical composition being principally determined by the geographical source of the sugar cane, the variety of cane and the method of processing.

Molasses and other products of the sugar refining process, such as the sugar mill mud, field trash/fibrated sugar cane tops, cane strippings and bagasse/pulp are complex mixtures of substances. Molasses and the other thick syrups and juices typically comprise lipids, phospholipids, protein, flavonoids, flavones, polyphenols, phytosterols, oligosaccharides, polysaccharides, peptides and proteins, minerals, organic acids, and mono and disaccharides.

### Beneficial Sugar Components

US patent application 2003 198694 teaches that antioxidant compounds that are beneficial for human health are present in sugar cane and sugar beet. These antioxidants include polyphenols and flavonoids and can be used in the production of functional food products.

These extracts may be used in a formulation strategy directed to reduction of glycaemic index (GI). GI is a system for classifying carbohydrate-containing foods according to how fast they raise blood-glucose levels inside the body. A food with a higher GI value raises blood glucose faster and is less beneficial to blood-sugar control that a food with a lower GI. For example, international patent application no WO2005/117608 discloses a method for lowering GI of a foodstuff by increasing the antioxidant content. This is typically achieved by adding extracts of sugar cane production waste streams and in-process products or other carbohydrates to the currently available highly refined sucrose products.

Polyphenols (or phenolics) are one of the classes of compounds present in sugar cane and are characterised by having a phenolic ring structure and two or more phenolic hydroxy groups. There are at least 8000 identified polyphenols in a number of subcategories such as anthocyanins and catechins. Natural polyphenols can range from simple molecules such as phenolic acid to large highly polymerized compounds such as tannins. Conjugated forms of polyphenols are the most common, and comprise various sugar molecules, organic acids and lipids (fats) linked with the phenolic ring structure. Differences in this conjugated chemical structure account for different chemical classifications and variation in the modes of action and health properties of the various phenols. Polyphenols present in sugar cane are considered to have a number of health benefits.

In the past, identification of a profile of desirable species (such as polyphenol) in extracts of sugar cane or sugar beet or sugar processing streams has been relatively difficult, requiring a range of wet chemical and spectroscopic techniques. This is principally due to the large number of phytochemicals present in sugar and their wide range of structures, making meaningful qualitative and quantitative analysis very difficult.

FIGS. 1 to 3 demonstrate the variation in the phytochemical content of different varieties of sugar cane using high pressure liquid chromatography (HPLC). It is this natural variation which leads to difficulties in producing sugar products containing consistent levels of phytochemicals.

There is thus a need for a method that can more routinely measure the levels of these phytochemicals in sugar products, especially where the phytochemical level is related to a particular functionality.

### SUMMARY OF THE INVENTION

It has now been found that NIR can be used to provide rapid quantitative and qualitative detection of species such as polyphenols, antioxidants, organic acids, colorants, polysaccharides, minerals, reducing sugars, policosanols, phytosterols, neutral lipids, phospholipids, emulsifiers, proteins and other phytochemicals which can be used to identify desired compositional profiles in sugar, sugar extracts, sugar processing or waste streams (including juice, bagasse, molasses, mill mud, dunder, strippings etc).

According to a first aspect of the invention, there is provided a process for the manufacture of sugar products having desired levels of specific phytochemicals comprising the steps of:
(a) preparing a primary sugar product;
(b) analysing the primary sugar product from step (a) using near infrared spectroscopy for its phytochemical profile;
(c) comparing the profile from step (b) with a reference phytochemical profile; and
(di) treating the primary sugar product, if required, to achieve a final sugar product having the desired levels of specific phytochemicals;
   or, alternatively
(dii) altering the preparation process in step (a), if required, to produce a primary sugar product having the desired levels of specific phytochemicals.

Preferably, the method permits rapid identification of sugar enriched with a level of polyphenols, antioxidants, organic acids, colorants, polysaccharides, soluble fibre, insoluble fibre, minerals, reducing sugars, policosanols, phytosterols, neutral lipids, phospholipids, emulsifiers, proteins or other phytochemicals that are desirable, both in terms of the types and quantities of each of these species.

When used herein, the term “sugar product” includes sugar, extracts from sugar cane, extracts from sugar processing or waste streams, and mixtures thereof.

The process of the invention is particularly useful for the manufacture of low GI sugar. In one embodiment, the primary sugar product is the standard crystalline raw or mill white sugar product (from cane or beet). This primary sugar product is then analysed using NIR to ascertain whether the level of each desired phytochemical species is sufficient for the sugar to be low GI. If there are not sufficient levels of the desired phytochemical species, then the primary sugar product will be treated by spray coating it with a molasses extract (for example, an extract as taught in international patent application nos WO2005/117608 and PCT/AU2007/001382) to increase the levels of the desired phytochemical species and lower the GI characteristics to form a low GI sugar. If the primary sugar product has the desired profile, then no treatment is required in step (d).

The method is also useful in producing sugar products which have a variety of different phytochemical level profiles to provide a food product having a specific functionality. For example, a sugar product having high antioxidant levels would be useful for food products aimed at conditions relating to oxidative damage. A sugar product having high fibre levels is another alternative. Such fibres can include soluble and insoluble fibres such as, but not limited to, celluloses, hemicelluloses and fructo-oligosaccharides. International patent application no PCT/AU2007/001382 discloses methods which can be used to prepare suitable extracts to be used in step (d) to achieve the desired phytochemical profile.

The preparation of a primary sugar product in step (a) can be by any method known to the person skilled in the art. Typically, the primary sugar product will be manufactured using standard sugar mill and refinery methods. Preferably, the manufacture of the primary sugar product will be designed to maximise the likelihood that the base sugar product will have the desired phytochemical profile. In one preferred embodiment, the manufacture of the primary sugar product will include the addition of affination syrup or a molasses extract to increase the levels of the desired phytochemical species. However, the raw materials from which sugar is made often varies in its composition depending on the crop, therefore it will still be necessary to analyse each batch.

The NIR analysis can occur on-line or off-line. Typically, when the method of the first aspect of the present invention is used in respect of on-line sugar processing streams, the scanning head is mounted adjacent a continuous stream of processed material. With respect to off-line measurements, typically, the samples are manually collected from the sugar processing stream and the NIR measurements are then made in a laboratory or similar facility.

Preferably, the NIR analysis occurs on-line. Typically, the NIR analysis in step (b) comprises:
(i) mounting a scanning head adjacent the extract (off-line) or processing stream (in-line), the scanning head comprising a remote light source and reflected light gathering and transmission apparatus;
(ii) using a monochromator of a near infrared spectrophotometer to resolve the reflected light into light of a discrete wavelength;
(iii) accessing a database containing a reference calibration equation linking absorption characteristics by discrete wavelengths with the quantified presence of each of the species of interest;
(iv) using a computer to create a profile of each of the species of interest by application of the calibration equation to the obtained spectrum for the extract or processing stream, and
(v) comparing the profile created with desired profile parameters stored in the database to identify the desired profile of the one or more species.

The treatment of the primary sugar product in step (di) can occur by any known method. Typically, steps (b) and (c) will involve the use of a computer which is programmed to instruct a spraying device to spray a substance onto the primary sugar product to increase the content of desired species. Typically, the treatment in step (di) will involve spraying onto the primary sugar product a sugar cane extract rich in a range of individual or mixed compounds such as polyphenols, antioxidants, organic acids, colorants, polysaccharides, soluble fibre, insoluble fibre, minerals, reducing sugars, policosanols, phytosterols, neutral lipids, phospholipids, emulsifiers or proteins. A suitable sugar cane extract for use in this treatment is described elsewhere such as international patent application nos WO 2005/117608 and PCT/AU2007/001382.

The alteration of the preparation of the primary sugar product using a manual or automatic feedback loop in step (dii) will involve manipulation of methods known to persons skilled in the art. Such methods include manipulation of the processing parameters, addition of chemical species or introducing one or more physical methods such as solvent extraction, size exclusion processing or ion exchange chromatography into the process. Preferably, the primary sugar manufacture process includes the addition of a phytochemical extract into any convenient stage in the process, such as for example, at the point of centrifuging or the screw conveyor. In a preferred embodiment, the syrup or powder derived from a sugar processing stream can then be applied into the sugar processing. Typically, the results from the NIR analysis on the final raw sugar (previously treated with a polyphenol syrup spray) as it leaves the drier are used as a process control tool to regulate the amount (percentage) of syrup added at the fugal or screw conveyor stage in order to achieve the desired levels of specific phytochemicals.

The desired profile of phytochemicals will vary depending upon, among other factors, the intended use, the desired antioxidant potential, mode of delivery (ie how and into which food or beverage or pharmaceutical the derivatives are ultimately incorporated), the intended therapeutic use as well as other factors known to those skilled in the art.

In a preferred embodiment where the final sugar product is a low GI sugar product, the desired profile measured includes one or more of the following species in the following amounts:

| Component | Range | Preferably |
|---|---|---|
| Sucrose (%) | 98.5-99.5 | 98.8-99.2 |
| Polyphenols (CE/100 g) | 20-45 | 25-40 |
| Antioxidant (mg GAE/100 g) | 5-14 | 6-12 |
| Organic Acids (mg/100 g) | 18-36 | 22-32 |
| (t-aconitic acid) | | |
| Total Minerals (mg/kg) as Ca, Mg, | 340-900 | 500-750 |
| K, Na | | |
| Color (ICUMSA) | 400-1600 | 900-1400 |
| Glycemic Index | 40-54 | 50-54 |
| Reducing Sugars (%) | 0-5 | 0-0.3. |

In the above table, CE stands for catechin equivalents, GAE stands for gallic acid equivalents and ICUMSA stands for International Commission for Uniform Methods of Sugar Analysis.

More preferably, the low GI sugar product has a profile of specific minerals as follows:

| | Range (% of | |
|---|---|---|
| Component | Total Minerals) | Preferably (mg/kg) |
| Potassium | 40-80% | 300-400 |
| Calcium | 25-35% | 180-380 |
| Magnesium | 4-8% | 20-50 |
| Sodium | 4-9% | 20-40 |
| Ratio between | | 5-7:0.8-1.2:8-12:0.0-1.3 |
| Ca/Mg/K/Na | | |

The principle of the invention can be used to provide rapid quantitative and qualitative detection of phytochemical levels on carriers other than sugar and using phytochemicals from sources other than sugar cane.

According to a second aspect of the invention, there is provided a process for the manufacture of a food product comprising
(a) preparing a base phytochemical carrier;
(b) analysing the base phytochemical carrier using near infrared spectroscopy;
(c) comparing the profile from step (b) with a reference profile; and
(di) treating the base phytochemical carrier, if required, to achieve a food product having the desired levels of phyto chemicals;
   or alternatively
(dii) altering the preparation process in step (a), if required, to produce a base phytochemical carrier having the desired levels of phytochemicals.

The base phytochemical carrier can be selected from fibre (insoluble sources such as bagasse and soluble sources), flour, cereals, dairy and other food products. The preparation step (a) and/or the treatment step (di) will typically involve adding phytochemicals to the carrier using various application processes, including but not limited to spray coating and agglomeration, to provide the desired functionality in the finished food product.

Depending upon the finished food application, an emulsifier or solubilizing compound may also be incorporated into the base phytochemical carrier to assist with dissolution of phytochemicals in the food matrix, delivery into the gastrointestinal tract following consumption, and dispersion of the sugar/fibre complex or to improve bioavailability of the added compounds.

Typical sources include, but are not limited to, cocoa beans and cocoa processing by-products, tea and tea processing waste streams, cocoa pod husks and shells, coffee beans, coffee waste, grape pomice, cereals (eg barley, buckwheat, corn, millets, oats, rice, rye, sorghum, wheat), legumes (eg beans and pulses), nuts (eg almonds, betel nuts, cashew nuts, hazelnuts, peanuts, pecans, walnuts), oilseeds (eg rapeseed, canola, soybeans, borage, cottonseed, evening primrose, flaxseed, sesame seeds, sunflowers, olive oil, palm oil, rice bran oil), fruits (eg berries, drupes, pomes, tropical fruits), vegetables (eg carrots, onions, parsnips, potatoes, beetroot, sweet potato, asparagus, celery, endive, lettuce, spinach, avocado, tomato, pepper), beverages (eg tea, coffee, cocoa, beer, wine, cider) and herbal products (eg Echinacea, ginseng, ginkgo biloba, St John's Wort, valerian, kava kava, saw palmetto, black cohosh, Devil's Claw, goldenseal, hawthorn, ginger, liquorice, milk thistle).

According to a third aspect of the invention, there is provided a process for the manufacture of an extract of sugar cane comprising
(a) preparing a first extract of sugar cane;
(b) analysing the first extract of sugar cane using near infrared spectroscopy;
(c) comparing the profile from step (b) with a reference profile; and
(di) treating the first extract of sugar cane with further extraction processes, if required, to achieve the desired levels of phyto chemicals;
   or alternatively
(dii) altering the preparation process in step (a), if required, to produce an extract of sugar cane having the desired levels of phytochemicals.

It has now also been established that there is a correlation between electrical conductivity (EC) and polyphenols content which can be used for online or offline measurement of polyphenols levels in sugar, sugar cane, extracts from sugar cane and from sugar processing or waste streams as disclosed in international patent application nos WO 2005/117608 and PCT/AU2007/001382 (including juice, bagasse, molasses, mill mud, dunder, strippings, affination syrup etc).

According to a fourth aspect of the invention, there is provided a process for the manufacture of an extract of sugar cane comprising:
(a) preparing a first extract of sugar cane;
(b) analysing the first extract of sugar cane using electrical conductivity;
(c) comparing the value from step (b) with a reference value; and
(di) treating the first extract of sugar cane with further extraction processes, if required, to achieve the desired levels of phytochemicals;
   or alternatively
(dii) altering the preparation process in step (a), if required, to produce an extract of sugar cane having the desired levels of phytochemicals.

The extract of sugar cane and the processes for its manufacture are known to those skilled in the art. Examples of extracts and the processes for their manufacture are disclosed in international patent application nos WO 2005/117608 and PCT/AU2007/001382.

Alteration of the process used to obtain the extract can change the profile of phytochemicals in the extract and the method of the second aspect of the present invention allows rapid identification of a desired level and thus the ability to control the quantity and nature of the downstream products if the extract is added.

The extract can be derived from any product derived from sugar cane including the sugar cane milling process, the sugar cane refining process to make sugar, and other processes using sugar cane products such as the manufacture of ethanol from molasses as part of the manufacture of rum. The extract can be derived from the raw materials, in-process products, by-products, final products and waste streams. For example, the sugar cane derived product may be the feed stream of raw sugar cane juice, clarified juice and concentrated juice syrup, treacle, molasses (obtained from a primary mill or refinery), golden syrup, brown sugar, bagasse, biodunder, field trash, growing tips, pulp, cane strippings, pith and mill mud. Preferably, the extract is derived from molasses.

In one preferred embodiment, the polyphenols in the extract of cane sugar are selected from the group consisting of p-coumaric acid, ferulic acid, syringic acid, caffeic acid, chlorogenic acid, (−)epicatechin, apigenin, (+)catechin, quercetin, diosmin, rutin and mixtures thereof.

The extract of sugar cane may comprise some carbohydrates which improves its taste whilst still maintaining its GI lowering characteristics. Typically, the extract comprises carbohydrates such as monosaccharides, disaccharides, oligosaccharides and both soluble and insoluble polysaccharides. The extract may also contain xylan derived mono, di, tri and oligosaccharides, such as xylobiose, xylotriose and xylose. The extract may include carbohydrates having GI increasing characteristics such as sucrose and glucose. However, the amount of GI increasing carbohydrates in the extract is not sufficient to detract significantly from the GI reducing characteristics of the extract as a whole. Further, the extract can comprise some carbohydrates and maintain its usefulness for applications such as body composition redistribution as disclosed in international patent application no PCT/AU2006/000769.

The extract of sugar cane may comprise minerals including mineral complexes. Typically, the minerals are selected from magnesium, potassium, iron, manganese, aluminium, zinc, calcium, sodium and mixtures thereof. Other minerals which may be present include anions such as nitrate, phosphate, sulphate and chloride.

The extract of sugar cane may comprise organic acids. Typically, the organic acids are selected from the group consisting of c-aconitic acid, citric acid, phosphoric acid, gluconic acid, malic acid, t-aconitic acid, succinic acid, lactic acid and mixtures thereof.

The extract can be used in a syrup form as an additive to white refined sugar from cane or beet to deliver the phytochemicals in the right range to change the refined sugar to a low GI sugar. The various process streams in the sugar refinery, such as affination syrup, or other washings from ion-exchange or activated charcoal columns, can also be used to adjust the phytochemical levels in the sugar during the refining process to produce a low GI sugar. Depending on the variety of sugar cane and the manufacturing process, in some instances, it will not be necessary to add extra phytochemicals to the sugar product to produce a low GI sugar.

### Uses of the Product of the Method of the Present Invention

The method of the present invention can be used to provide new products which are economically useful and can be used in a wide variety of applications.

The products of the process of the present invention may be incorporated directly and without further modification into a food, nutraceutical or beverage by techniques such as mixing, infusion, injection, blending, dispersing, conching, emulsifying, immersion, spraying, agglomeration and kneading. Alternatively, the products may be applied directly onto a food or food matrix into a beverage by the consumer prior to ingestion.

As used herein, the term “food”, “foodstuffs” or “food product” includes any edible product, such as but not limited to confectioneries, supplements, snacks (sweet and savoury), cocoa & coffee-containing foods, flavours, beverages (including instant beverages, pre-mixes), dietary supplements and formulations including supplements used in animal health and nutrition, dairy products eg: milk, yogurt, ice-cream, baked products, and food seasonings.

The products of the present invention may be incorporated into foods, beverages and nutriceuticals, including, without limitation, the following:
Dairy Products—such as cheeses, butter, milk and other dairy beverages, spreads and dairy mixes, ice cream and yoghurt;
Fat-Based Products—such as margarines, spreads, mayonnaise, shortenings, cooking and frying oils and dressings;
Cereal-Based Products—comprising grains (for example, bread and pastas) whether these goods are cooked, baked or otherwise processed;
Confectioneries—such as chocolate, candies, chewing gum, desserts, non-dairy toppings, sorbets, icings and other fillings;
Enteral and parenteral products,
Sports nutrition products including powders, pre-mixes, juices, energy bars, isotonic drinks and gelatine, starch based or pectin jellies;
Beverages—whether hot or cold (coffee, tea, cocoa, cereal, chicory and other plant extract based beverages), alcoholic or non-alcoholic and including colas and other soft drinks, juice drinks, dietary supplement, instant pre-mixes and meal replacement drinks; and
Miscellaneous Products—including eggs and egg products, processed foods such as soups, pre-prepared pastas.

### Low GI Food Products

In a particularly preferred application, the processes of the present invention may be utilized to manufacture products to be used in a strategy directed to the reduction of GI. The processes of the invention can be used to prepare the low GI products, for example, as disclosed in international patent application no WO05/117608, but with the additional benefit of a specific phytochemical content.

For a low GI product, it is preferable to have low glucose levels. The glucose content of the extract of a sugar cane product can be reduced using enzymes such as glucose oxidase (GO) which digest glucose. It will be known by those skilled in the art that a combination of glucose oxidase and catalase is typically used to ensure that any hydrogen peroxide produced is removed, and that the oxygen generated is then used by the GO to further reduce glucose levels. The method of the invention may also incorporate any other method to reduce glucose and other products which is then reincorporated in the manufacturing process to reduce the GI of the sugar product. This may include, but is not limited to, fermentation, or encouragement of glucose digestion through other chemical, and/or thermal reactions prior to, during or after the ultrafiltration and ion exchange processes.

The method of the invention may also be used to prepare products for use in the methods and products disclosed in international patent application no PCT/AU2006/00076.

### BRIEF DESCRIPTION OF THE FIGURES

FIG. A illustrates an NIR system which could be used to implement the process according to one embodiment of the invention.

FIG. 1 shows the phytochemical composition as measured by HPLC of a first variety of sugar cane.

FIG. 2 shows the phytochemical composition as measured by HPLC of a second variety of sugar cane.

FIG. 3 shows the phytochemical composition as measured by HPLC of a third variety of sugar cane.

FIG. 4 shows the comparative conductivity vs. polyphenols (catechin equivalents) results for a range of sugars as described in Example 2.

FIG. 5 shows the comparative color vs. polyphenols (catechin equivalents) results for a range of sugars as described in Example 2.

FIG. 6 shows the statistical correlation of catechin vs. conductivity results for Example 2.

FIG. 7 shows the statistical correlation of catechin equivalence vs. color results for Example 2.

FIG. 8 shows the Pol (Sucrose) % Sugar NIR Calibration Plot from Example 3.

FIG. 9 shows the Moisture % Sugar NIR Calibration Plot from Example 3.

FIG. 10 shows the Ash % Sugar NIR Calibration Plot from Example 3.

FIG. 11 shows the Sugar Colour NIR Calibration Plot from Example 3.

FIG. 12 shows the Reducing Sugars (Lane and Eynon) NIR Calibration Plot from Example 3.

FIG. 13 shows the Conductivity Ash NIR Calibration Plot from Example 3.

FIG. 14 shows the Fine Grain (% by mass less than 600 microns) NIR Calibration Plot from Example 3.

FIG. 15 shows the Total phenolics NIR calibration plot from Example 3.

FIG. 16 shows the Trans-Aconitic Acid NIR calibration plot from Example 3.

FIG. 17 shows the Anti-Oxidant Potential NIR calibration plot from Example 3.

FIG. 18 shows the Sugar Conductivity NIR calibration plot from Example 3.

FIG. 19 shows the Glucose % Sugar NIR calibration plot from Example 3.

FIG. 20 shows the Fructose % Sugar NIR calibration plot from Example 3.

FIG. 21 shows the Calcium in Sugar NIR calibration plot from Example 3.

FIG. 22 shows the Magnesium in Sugar NIR calibration plot from Example 3.

FIG. 23 shows the Sodium in Sugar NIR calibration plot from Example 3.

FIG. 24 shows the Potassium in Sugar NIR calibration plot from Example 3.

FIG. 25 shows the Iron in Sugar NIR calibration plot from Example 3.

FIG. 26 shows the Aluminium in Sugar NIR calibration plot from Example 3.

FIG. 27 shows the Manganese in Sugar NIR calibration plot from Example 3.

FIG. 28 shows the Zinc in Sugar NIR calibration plot from Example 3.

FIG. 29 shows the Chloride in Sugar NIR calibration plot from Example 3.

FIG. 30 shows the Sulphate in Sugar NIR calibration plot from Example 3.

FIG. 31 shows the Phosphate in Sugar NIR calibration plot from Example 3.

FIG. 32 shows the Sugar Filtrability NIR calibration plot from Example 3.

FIG. 33 shows the NIR Calibration Statistics from Example 3.

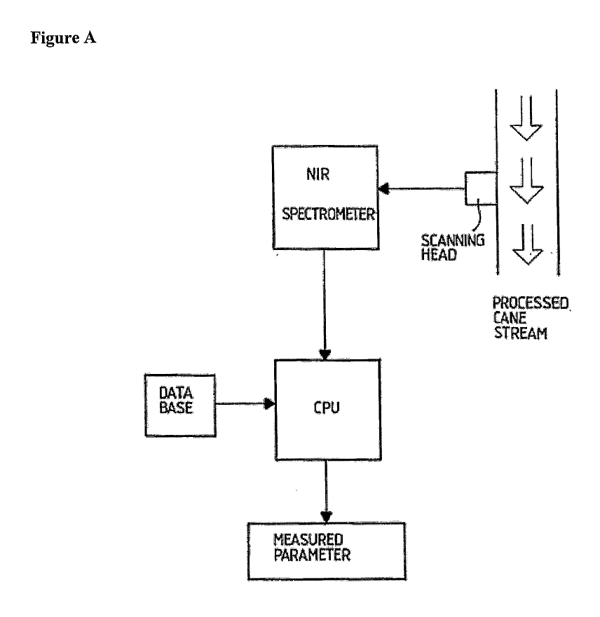

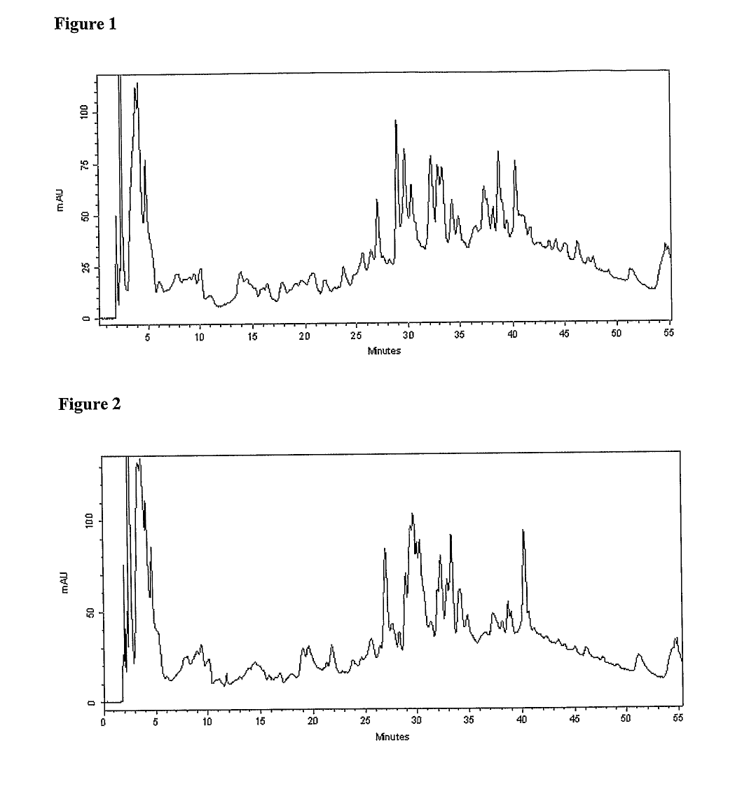

### DETAILED DESCRIPTION OF ONE EMBODIMENT OF THE INVENTION

### Near Infra Red Spectroscopy

Near infra red spectroscopy provides a rapid means of determination of a product's attributes. BSES Limited (BSES), an Australian sugar industry research organization has previously used NIR technology to develop a Cane Analysis System (CAS) capable of direct, real-time analysis of prepared or shredded cane for payment and process control purposes. Since implementation of the first NIR system in Australia, BSES have developed extensive databases necessary for the application of these calibration equations (Staunton S. P., Lethbridge P. T., Grimley S. C., Streamer R. W., Rogers J., Macintosh D. L., Online cane analysis by near infra-red spectroscopy. Proc. Aust. Soc. Sugar Cane Technol. 1999; 21:20-27) across different mills and growing regions and for additional substrates such as sugar and bagasse (Staunton S. P. & Wardrop K., Development of an online bagasse analysis system using NIR spectroscopy. International Sugar Journal, 2007, 109:482-488).

No evidence however previously existed of a correlation between NIR spectra and the polyphenol content of sugarcane or sugar. The present invention includes an NIR system for online/offline production of high antioxidant/low GI or functional sugars.

The Sugar Analysis System (SAS) used for the present invention comprises the following hardware and software.

| Hardware | Software |
|---|---|
| A FOSS Direct Light NIR | Operating System (eg Windows |
| spectrophotometer | XP) |
| A control computer | System software (‘SAScontrol’) |
| Uninterruptible powers supply (UPS) | developed by BSES to manage the |
| Allowance for network connection | whole system and, undertake |
| Instrument read head mounted on a | computations of predictions, |
| vibration damping arrangement | manages all communications and |
| designed for specific vibrational | provides system protection. It |
| frequencies and installed within a | incorporates at its core software |
| special sealed mounting enclosure for | supplied by FOSS. |
| attachment above the moving stream | Sample start/finish information is |
| of process sugar | essential for calibration, validation |
| Interpretive instrument mounted on | and spectral identification |
| special vibration damping | purposes |
| arrangement within the | Constituent calibration equations |
| main enclosure | |
| Stainless steel enclosure for the | Remote access software to |
| electronic components | provide for remote support of the |
| Enclosure fitted with lifting lugs and | system and execution of |
| mounting points for transport of the | diagnostics |
| read head arrangements on the side of | |
| the main enclosure | |

FIG. A is a schematic drawing of a typical system. The scanning head of the direct light reflectance system of a NIR monochromator class of spectrophotometer is positioned alongside or above a process stream to be analysed. Reflected light passes by way of fibre optics to the spectrophotometer where the light is broken into wavelengths over the range 400 to 2500 mm in steps of typically 2 nm. A spectrum of the absorption by the process stream by wavelength is produced for each scan of the sample. A database of calibration equations is stored for each parameter of interest such as fibre content in the process stream. This information is held available for access by the CPU. The database also stores the characteristics of the spectra used in deriving the calibration equations. An average spectrum is produced for each sample scan. The relevant sections of the spectrum for the calibration of interest are extracted and computed to deliver the measured parameter for the scan. The results form all the accepted scans of the relevant portion of the process stream are averaged for the prediction. The spectrum obtained is useable for as many parameters as calibrations are available. The CPU can reject a spectrum that does not conform to the set of spectra used to derive the calibration equation.

Constituent calibrations equations useful for implementation of the invention include:

| | |
|---|---|
| Pol (Sucrose) | Polyphenols |
| Moisture | Minerals |
| Sugar colour (ICUMSA) | Organic acids |
| Reducing sugars | Antioxidants |
| Ash (sulphitation) | Glucose |
| Conductivity ash | Fructose |
| Fine grain | |

### Electrical Conductivity

Electrical conductivity (EC) estimates the amount of total dissolved salts (TDS), or the total amount of dissolved ions in solution. EC is measured in microSiemens per centimeter (μS/cm) and is recorded using a sensor which consists of two metal electrodes that are exactly 1.0 cm apart and protrude into the solution. A constant voltage (V) is applied across the electrodes. An electrical current (I) flows through the solution due to this voltage and is proportional to the concentration of dissolved ions present—the more ions, the more conductive the solution which results in a higher measured electrical current. Distilled or deionized water has very few dissolved ions and so there is almost no current flow across the gap (low EC). Since the electrical current flow (I) increases with increasing temperature, the EC values are automatically corrected to a standard value of 25° C. and the values are then technically referred to as specific electrical conductivity. EC probes generally have fast response time, usually reaching 98% of full value in less than 5 seconds. Some conductivity probes use alternating current at its electrodes in order to prevent polarisation and electrolysis, so that solutions being tested are not fouled. Probes are usually epoxy coated to prevent corrosion of metal electrodes which obviously affects conductivity readings.

EC would thus be a convenient method for online or offline measurement of polyphenols levels in sugar. However, no evidence however currently exists for a correlation between EC and polyphenols.

### EXAMPLES

Various embodiments/aspects of the invention will now be described with reference to the following non-limiting examples.

### Example 1

Low GI sugar (GI between 50 and 54) was prepared in a primary sugar mill which had been converted to food grade status with an approved and audited food safety system.

The preparation comprised the following steps:
1. Sugar massecuite was washed in the fugals to a composition below the final desired range being targeted i.e. low color and low polyphenol levels. This was achieved by adjusting the amount of water used, the time and G force of the fugal. Allowance was made from time to time for compositional variations in incoming cane varieties, the day to day variations in the overall extraction, clarification and crystallization process.
2. In a new and separate food grade facility, molasses was extracted and purified to produce a concentrated and standardized (mg polyphenol/L) polyphenol syrup. This syrup (a dark t yellow colored liquor of between 60-70 Brix) was metered into the washed base sugar in the fugal using a spraying system.
3. The syrup treated washed sugar was dried as per standard operations in continuous rotary driers and moisture, polyphenol, color and sucrose levels were measured either on line or off line using NIR technology. A feedback loop of data from the NIR measurement device was linked to the syrup dosing/spray system in the fugal. This enabled the correct amount of syrup from step 2 to be delivered onto the washed sugar mass of step 1 so the final polyphenol level in the dried sugar was in the range of 25-40 mg PP/100 g sugar.

Table 1 sets out the compositional parameters for key species in the low GI sugar with equivalent parameters for raw sugar and white sugar prepared according to normal commercial sugar processing techniques.

| | | Low GI | |
|---|---|---|---|
| | Raw Sugar | sugar | White Sugar |
| Component | Range | Range | (Comparison) |
| Sucrose (%) | 97.4-99.3 | 98.8-99.2 | 99.6-99.7 |
| Polyphenols (CE/100 g) | 15-100 | 25-40 | 0 |
| Antioxidant (mg GAE/100 g | 4-30 | 6-12 | 0 |
| Organic Acids (mg/100 g) | 16-90 | 22-32 | 0 |
| Potassium (mg/kg) | 9-1800 | 300-400 | 0 |
| Calcium (mg/kg) | 4-450 | 180-380 | 0 |
| Magnesium (mg/kg) | 18-134 | 20-50 | 0 |
| Sodium (mg/kg) | 15-98 | 20-32 | 0 |
| Color (ICUMSA) | 60-4100 | 800-1350 | <200 |

As shown in the table, raw sugars vary significantly in composition and as a result many are deficient in bioactive phytochemicals such as polyphenols. Sugars at another end of the spectrum lack acceptable organoleptic qualities, are hygroscopic, highly colored and cannot be used commercially because of difficulty in bulk handling and their impact on finished foods.

The compositional requirement for low GI sugars are unique in that they meet the clinical performance requirement (GI<55) while still providing acceptable handling, organoleptic, color, hygroscopic, crystal size and hence solubility alone and in food matrices. Examples of low GI sugar products which have these properties will be sold under the trade marks WHOLEMEAL SUGAR™ or LOGICANE™.

When the washed raw sugar was treated with a spray solution to form the low GI sugar, no changes to the crystal size were observed as these had already been determined during the crystallization process. However, subtle changes to the crystal morphology were observed as the phytochemicals and sucrose in the syrup attached to the surface of the already formed crystals. In addition, the color of the sugar crystals increased slightly due to the increase in polyphenol levels. Other functionalities such as flowability and hygroscopicity were not changed as this is largely controlled by moisture levels which are in turn controlled in the drier post the spraying process. When carefully controlled, production of a concentrated polyphenol spray solution delivered a standardized dose of the phytochemical to achieve a reduction in GI without compromising other desirable parameters of the sugar.

### Example 2

This example investigates whether there is any correlation between electrical conductivity (EC) and polyphenol levels in sugar. If confirmed then this could be used as a colorimetric method for online and offline polyphenol assessment.

Chemicals: Folin-Ciocalteu reagent and (+)-catechin standard were purchased from Sigma-Aldrich (St Louis, Mo.). Sodium carbonate was obtained from Labsery (Melbourne, Australia) and 3-(N-morpholino)-propanesulphonic acid (MOPS) was from BDH Laboratory Supplies (Dorset, UK). All chemicals used were analytical grade.

Sample Collection: Raw sugar samples were obtained from Mossman Central Mill (MCM) during standard sugar production. At regular intervals over a two day period approximately 100 g of raw sugar was sampled from the finished product conveyor using screw capped plastic bottles.

Polyphenol Analysis: 40 g of raw sugar sample was accurately weighed into a 100 ml volumetric flask. Approximately 40 ml of distilled water was added and the flask agitated until the sugar was fully dissolved after which the solution was made up to final volume with distilled water. The polyphenol analysis was based on the Folin-Ciocalteu method (Singleton 1965) adapted from the work of Kim et al (2003). In brief, a 50 μl, aliquot of appropriately diluted raw sugar solution was added to a test tube followed by 650 μL of distilled water. A 50 μL aliquot of Folin-Ciocalteu reagent was added to the mixture and shaken. After 5 minutes, 500 μL of 7% Na₂CO₃ solution was added with mixing. The absorbance at 750 nm was recorded after 90 minutes at room temperature. A standard curve was constructed using standard solutions of catechin (0-250 mg/L). Sample results were expressed as milligrams of catechin equivalent (CE) per 100 g raw sugar.

Colour Analysis: Colour was analysed according to the BSES Standard Analytical Method 33 (2001). In brief, 20 g of raw sugar was accurately weighed into a 100 ml volumetric flask; approximately 50 ml of distilled water was added and the flask agitated until the sugar dissolved. 10 mls of 0.2M MOPS buffer solution (pH 7) was added to flask and the solution made to up to the final volume with distilled water. A reference solution was made by the addition of 10 ml MOPS buffer to a 100 ml volumetric flask which was made up the mark with distilled water. Each sample solution and reference solution was filtered using a 0.8 μm prefilter connected to a 0.45 μm membrane filter (Millipore, Millex HA). Absorbance of the filtered sugar solution was measured at 420 nm using the reference solution as the blank. The ICUMSA colour was calculated using the following calculation:

ICUMSA colour=(A420/concentration in g/ml)×1000

Conductivity Measurement: A 20 g sample of raw sugar was accurately weighed into a 100 ml volumetric flask and the solution was made up to the mark with distilled water. The conductivity of the 20% solution was measured using a HANNA conductivity meter (Model H19812-5) standardized using a HANNA 1413 μS/cm calibration standard and the results were expressed as microsiemens per centimetre.

### Results:

Table 2 sets out the comparison of raw sugar conductivity (μS/cm), polyphenol content (mg CE/100 g) and colour assessments.

| | | | mg | | |
|---|---|---|---|---|---|
| | | | CE/ | Conductivity | Colour |
| | Date | Sample | 100 g | μS/cm | ICUMSA |
| Non FG | Oct. 23, 2007 | 0815 | 20.97 | 150 | 750 |
| | Oct. 23, 2007 | 0830 | 25.82 | 190 | 930 |
| Start FG | Oct. 23, 2007 | 0845 | 21.31 | 140 | 960 |
| | Oct. 23, 2007 | 0900 | 19.71 | 130 | 870 |
| | Oct. 23, 2007 | 0915 | 18.53 | 130 | 915 |
| Finish FG | Oct. 23, 2007 | 0930 | 24.05 | 180 | 1045 |
| New pan | Oct. 23, 2007 | 1050 | 16.57 | 110 | 620 |
| | Oct. 23, 2007 | 1105 | 15.88 | 100 | 665 |
| | Oct. 23, 2007 | 1115 | 17.87 | 120 | 710 |
| | Oct. 23, 2007 | 1120 | 24.02 | 160 | 1100 |
| | Oct. 23, 2007 | 1120 | 19.45 | 120 | 785 |
| | | Bucket | | | |
| | | elev | | | |
| | Oct. 23, 2007 | 1130 | 18.43 | 110 | 815 |
| | Oct. 23, 2007 | 1140 | 18.87 | 100 | 760 |
| finish pan | Oct. 23, 2007 | 1150 | 17.73 | 100 | 625 |
| new pan | Oct. 23, 2007 | 1200 | 22.53 | 130 | 750 |
| new pan | Oct. 23, 2007 | 1210 | 22.84 | 130 | 735 |
| | Oct. 23, 2007 | 1540 | 20.57 | 120 | 715 |
| | Oct. 23, 2007 | 1600 | 19.14 | 130 | 780 |
| | Oct. 23, 2007 | 1610 | 20.22 | 130 | 865 |
| | Oct. 23, 2007 | 1620 | 22.30 | 140 | 990 |
| | Oct. 23, 2007 | sieve | 34.83 | 290 | 1600 |
| | | fines | | | |
| | Oct. 24, 2007 | 0900 | 26.80 | 190 | 1135 |
| | Oct. 24, 2007 | 0910 | 24.43 | 160 | 1065 |
| | Oct. 24, 2007 | 0920 | 24.22 | 170 | 1145 |
| | Oct. 24, 2007 | 0930 | 23.62 | 180 | 1140 |
| | Oct. 24, 2007 | 0940 | 22.69 | 160 | 1050 |
| | Oct. 24, 2007 | 1130 | 31.47 | 210 | 1495 |
| | Oct. 24, 2007 | 1140 | 32.19 | 220 | 1560 |
| Start FG | Oct. 24, 2007 | 1150 | 31.58 | 210 | 1545 |
| | Oct. 24, 2007 | 1310 | 27.05 | 190 | 1130 |
| | Oct. 24, 2007 | 1320 | 28.46 | 210 | 1250 |
| | Oct. 24, 2007 | 1330 | 28.53 | 210 | 1080 |
| | Oct. 24, 2007 | 1420 | 37.71 | 300 | 1695 |
| Stop FG | Oct. 24, 2007 | 1440 | 41.72 | 310 | 1750 |
| | Oct. 24, 2007 | 1450 | 41.97 | 300 | 1725 |
| | Oct. 24, 2007 | 1505 | 34.85 | 270 | 1485 |
| | Oct. 24, 2007 | 1515 | 28.26 | 210 | 1280 |
| | Oct. 24, 2007 | 1520 | 29.96 | 200 | 1295 |

Table 3 sets out the Raw sugar colorimetric assay results (750 nm absorbance) and calculation of catechin equivalent (CE) in mg/100 g.

| | | | AV | mg Catechin/ | Weight raw | Volume of | mg CE/ |
|---|---|---|---|---|---|---|---|
| | | Av | A750nm - | mL from std | sugar | sample | 100 g of |
| Code # | Sample | A750 nm | blank | plot | sample (g) | (mL) | raw sugar |
| 1 | 23/10/07 1540 | 0.4340 | 0.3673 | 0.082 | 40.0169 | 100 | 20.571 |
| 2 | 23/10/07 1600 | 0.4115 | 0.3448 | 0.077 | 40.1674 | 100 | 19.141 |
| 3 | 23/10/07 1610 | 0.4280 | 0.3613 | 0.081 | 40.0006 | 100 | 20.216 |
| 4 | 23/10/07 1620 | 0.4625 | 0.3958 | 0.089 | 40.0392 | 100 | 22.297 |
| 5 | 23/10/07 fines | 0.6585 | 0.5918 | 0.139 | 40.0065 | 100 | 34.827 |
| 6 | 24/10/07 0900 | 0.5350 | 0.4683 | 0.107 | 40.0585 | 100 | 26.796 |
| 7 | 24/10/07 0910 | 0.4975 | 0.4308 | 0.098 | 40.0781 | 100 | 24.435 |
| 8 | 24/10/07 0920 | 0.4935 | 0.4268 | 0.097 | 40.0252 | 100 | 24.218 |
| 9 | 24/10/07 0930 | 0.4835 | 0.4168 | 0.094 | 39.9965 | 100 | 23.615 |
| 10 | 24/10/07 0940 | 0.4685 | 0.4018 | 0.091 | 39.9934 | 100 | 22.691 |

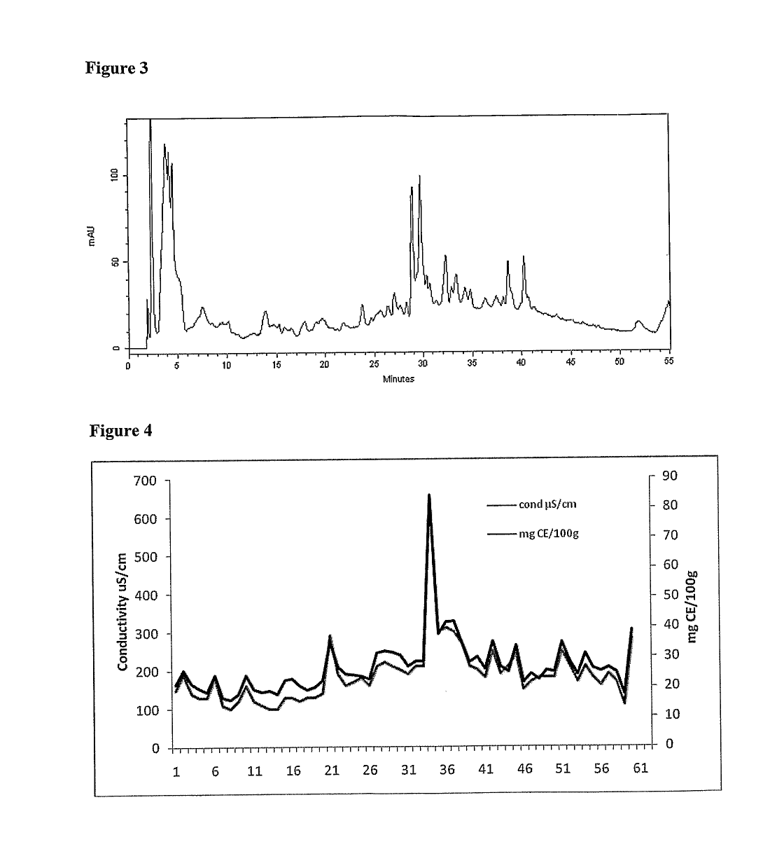

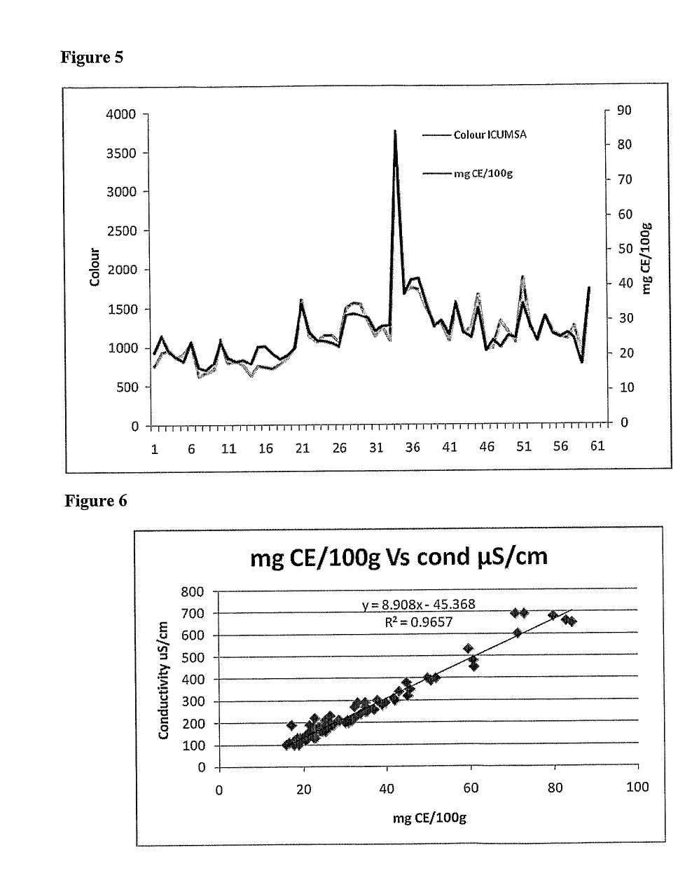

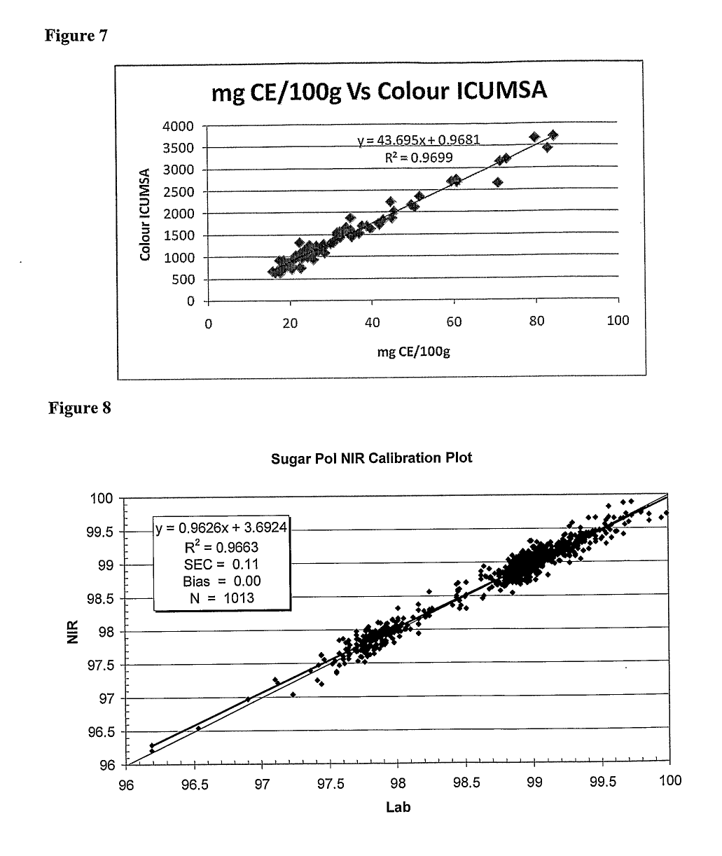

The results are further illustrated in FIGS. 4 to 7 and 18. It is clear from these figures that the sample points are all close to the linear plot and that the graphs are almost identical.

### Conclusion:

A surprisingly statistically significant correlation exists between EC, ICUMSA sugar color and polyphenol content. This method is therefore useful for a rapid on-line and/or offline measuring tool for QA/QC purposes in making sugar containing higher polyphenol levels. The method can readily be adapted and integrated into current industrial methodologies and process control systems.

### REFERENCES

Bureau of Sugar Experiment Stations (BSES) 2001. Laboratory Manual for Australian Sugar Mills Volume 2, Method 33, BSES Brisbane.
Kim D.-O., Jeong S. W. and Lee C. Y (2003). Antioxidant capacity of phenolic phytochemicals from various cultivars of plums. Food Chemistry, 81, (3) 321-326.
Singleton, V. L. and Rossi, J. A (1965). Colorimetry of total phenolics with phosphomolybdic-phosphotungstic acid reagents. Am. J. Enol. Vitic. 16, 144-158.

### Example 3

This example investigates the use of near infrared (NIR) spectroscopic methods to predict the quantities of polyphenolics, organic acids and minerals in sugar. This information can then be used to develop a suitable NIR method for online and offline polyphenol assessment for manufactured low GI sugars and polyphenol extracts.

Constituent equations developed in this example include:
Polyphenols (eg feulic acid)
Minerals
Organic acids (eg aconitic acid)
Antioxidants
Glucose
Fructose

The equations are purpose developed for the BSES system but with alteration, may be used in other NIR systems including for example, laboratory instruments which could be used in an offline situation.

### Results:

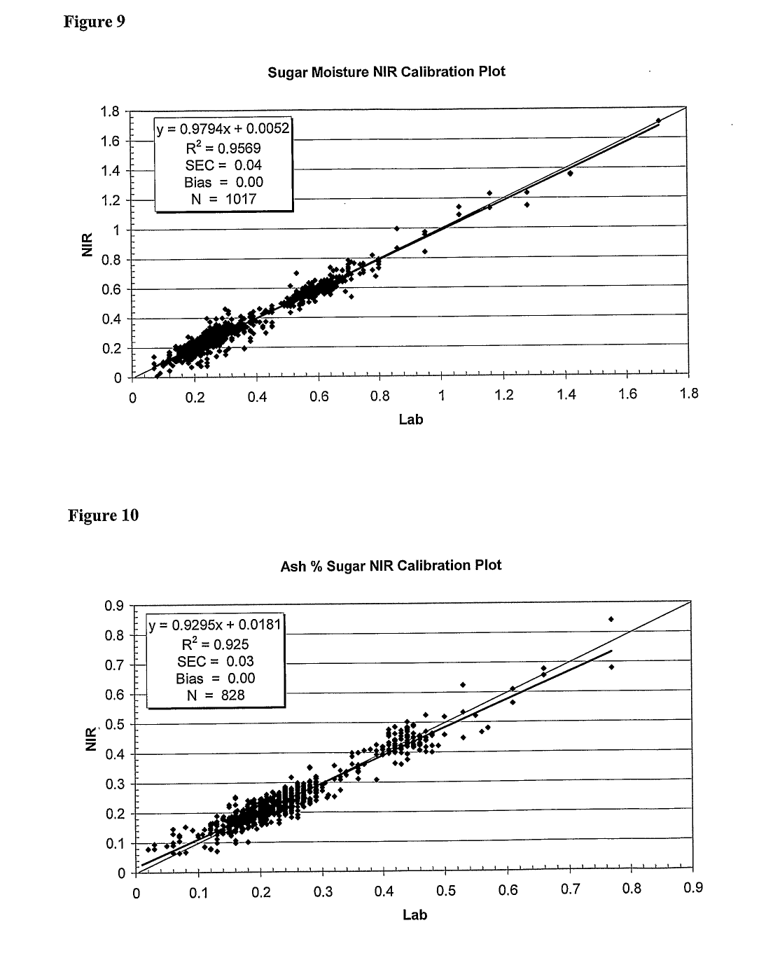

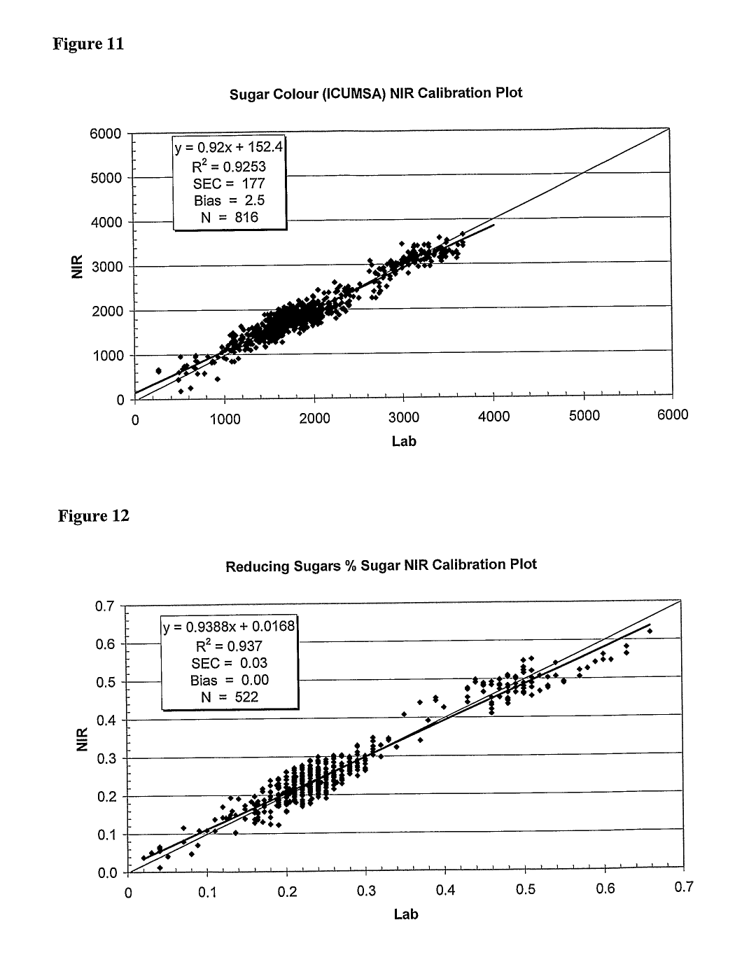

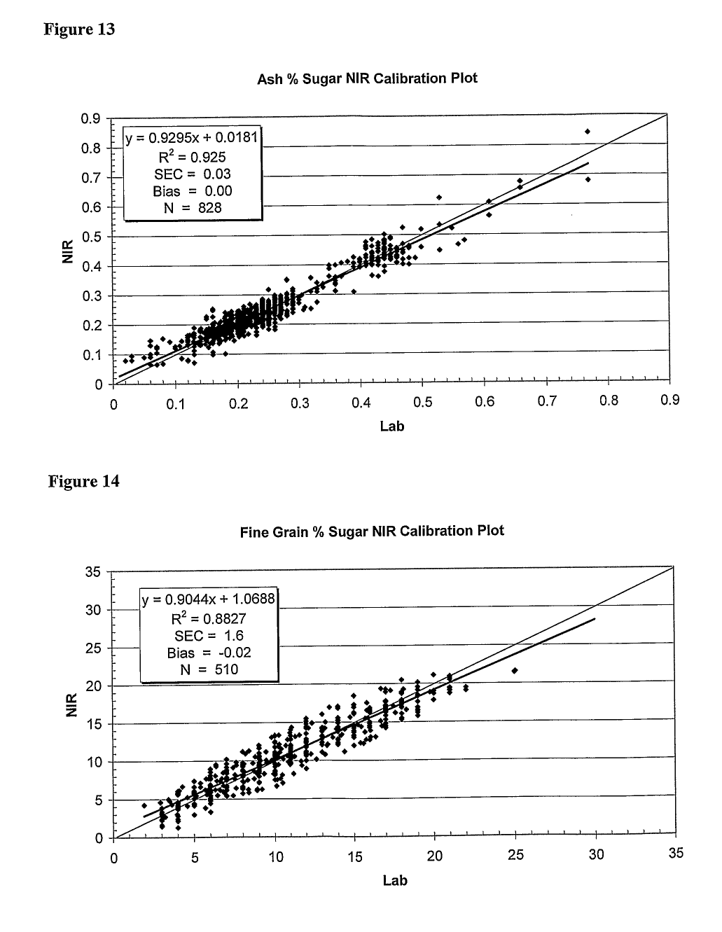

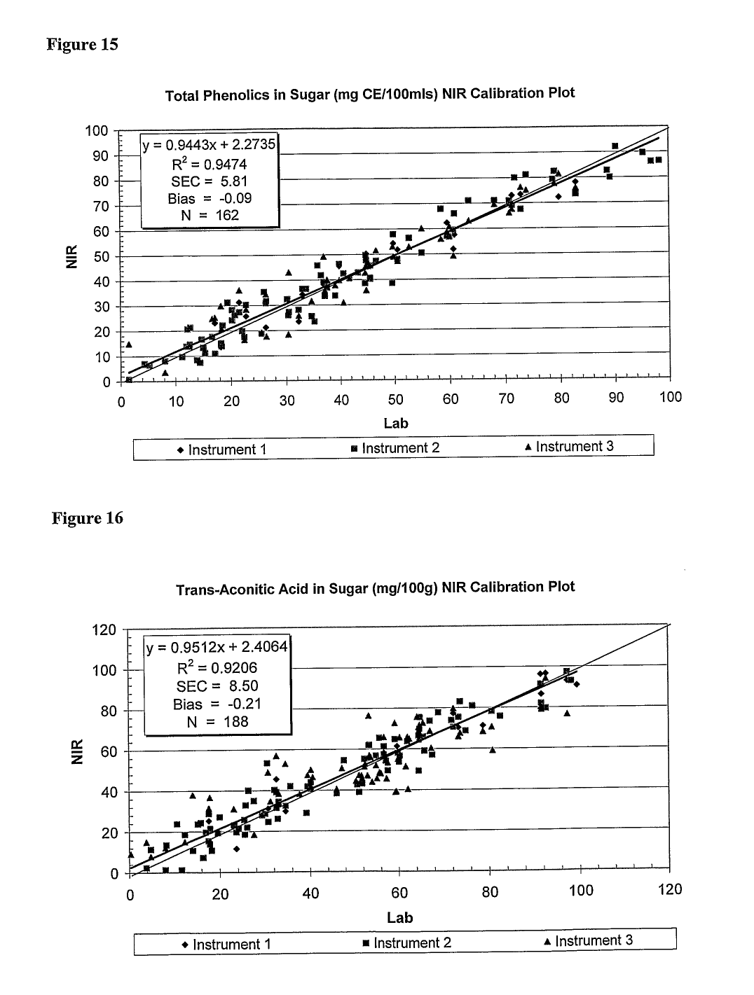

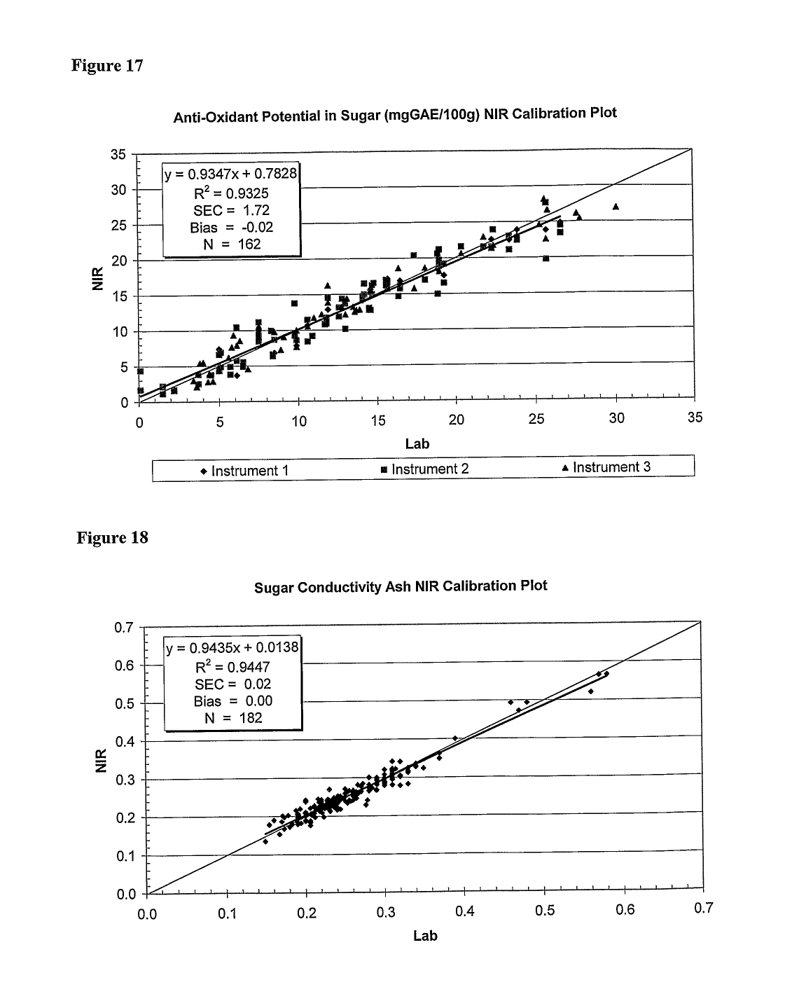

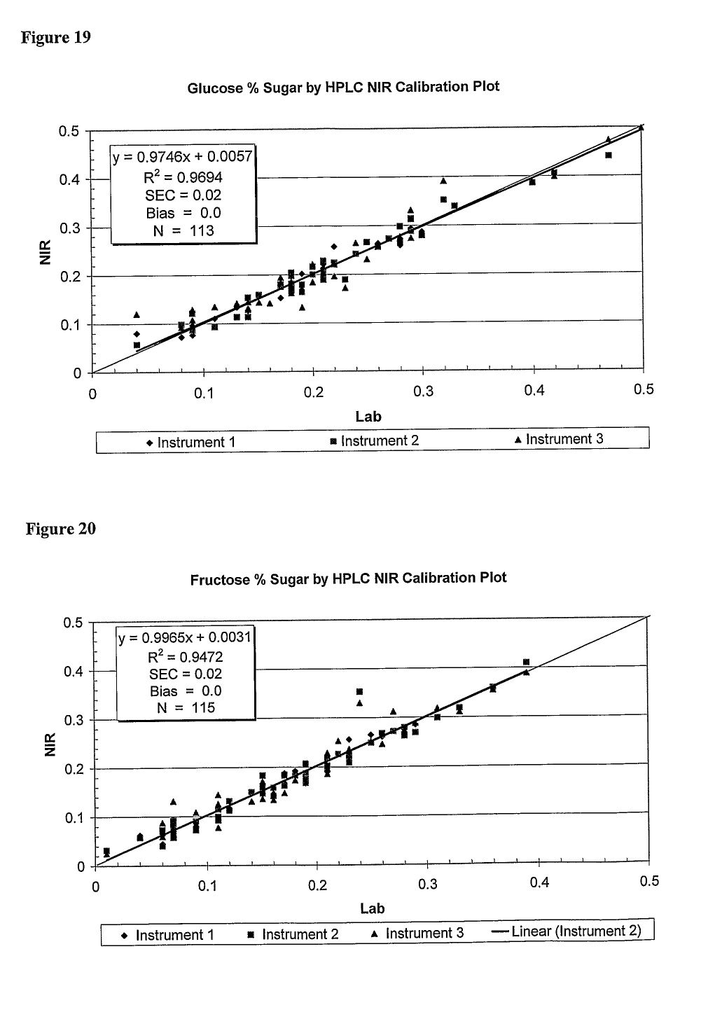

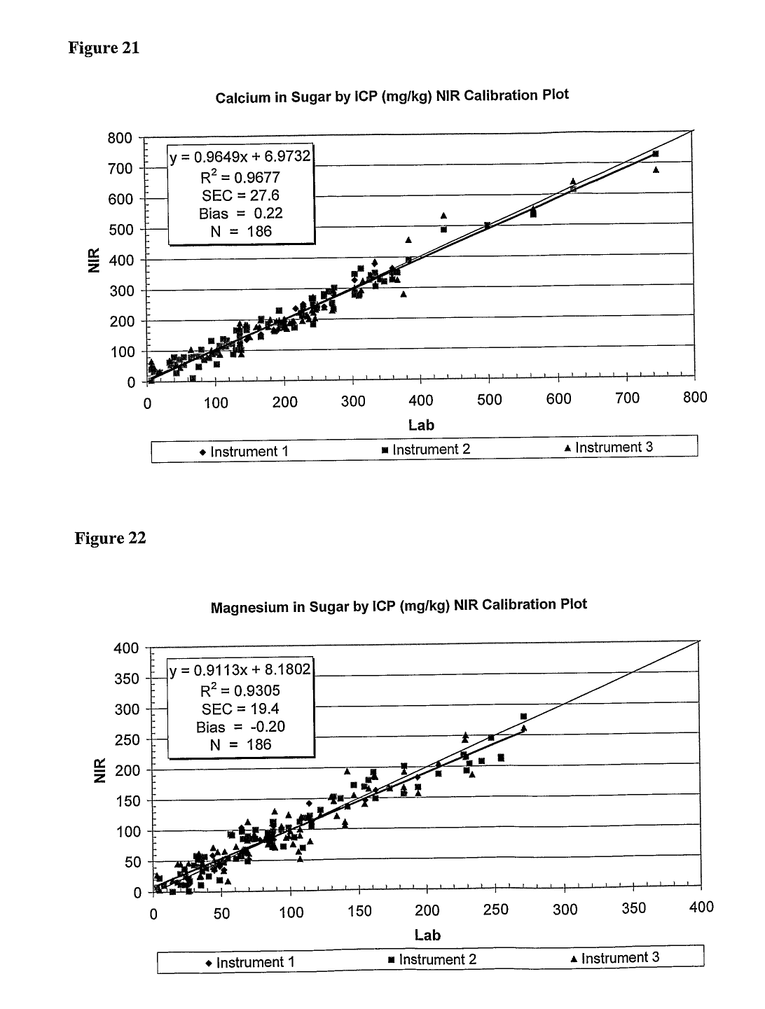

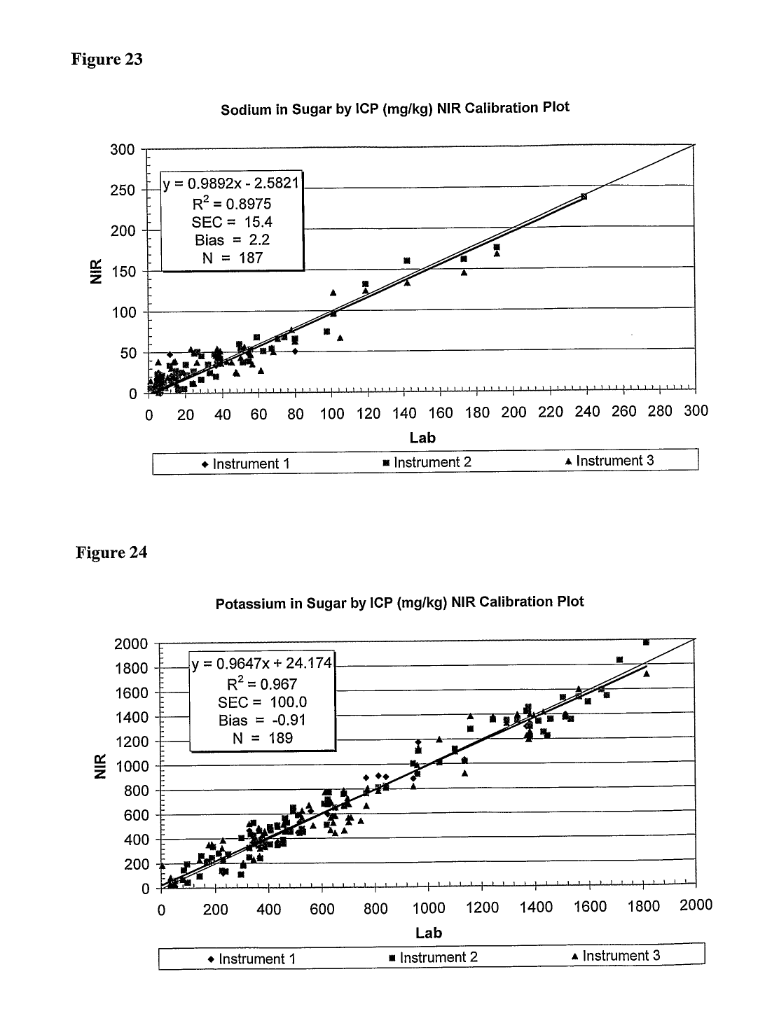

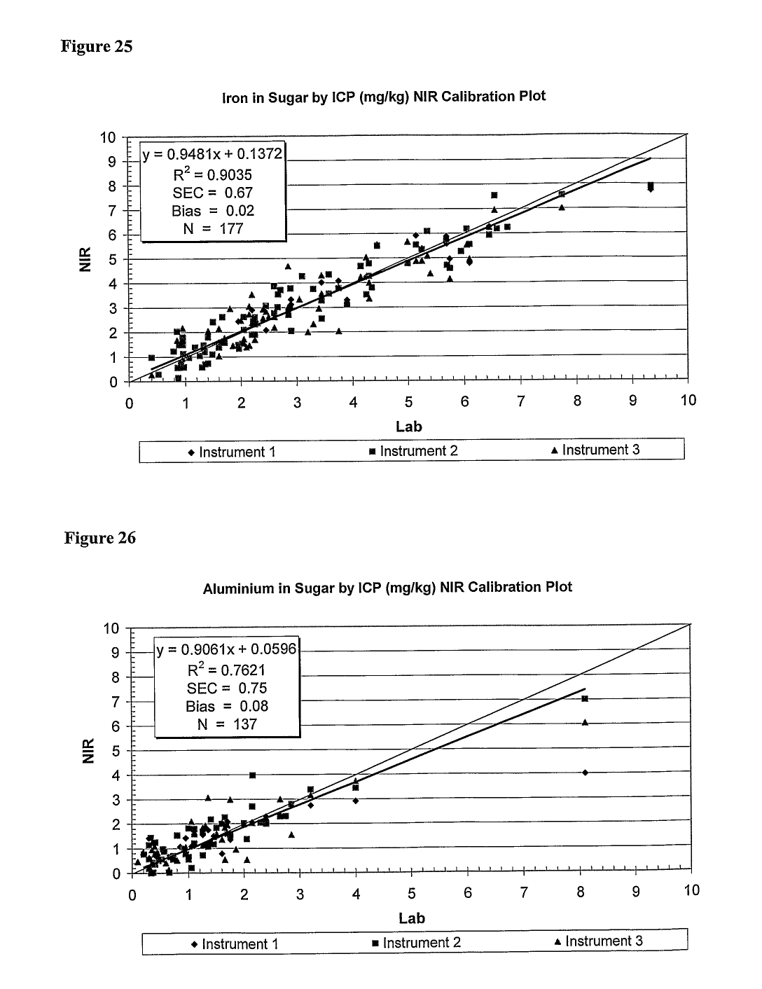

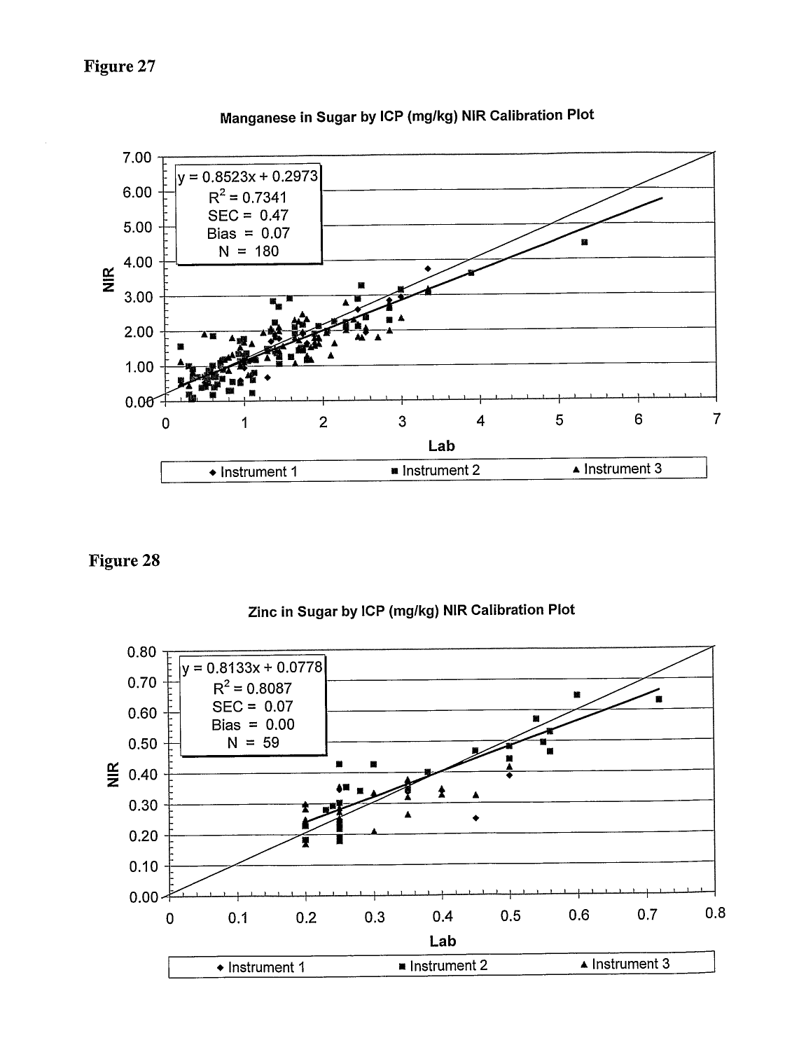

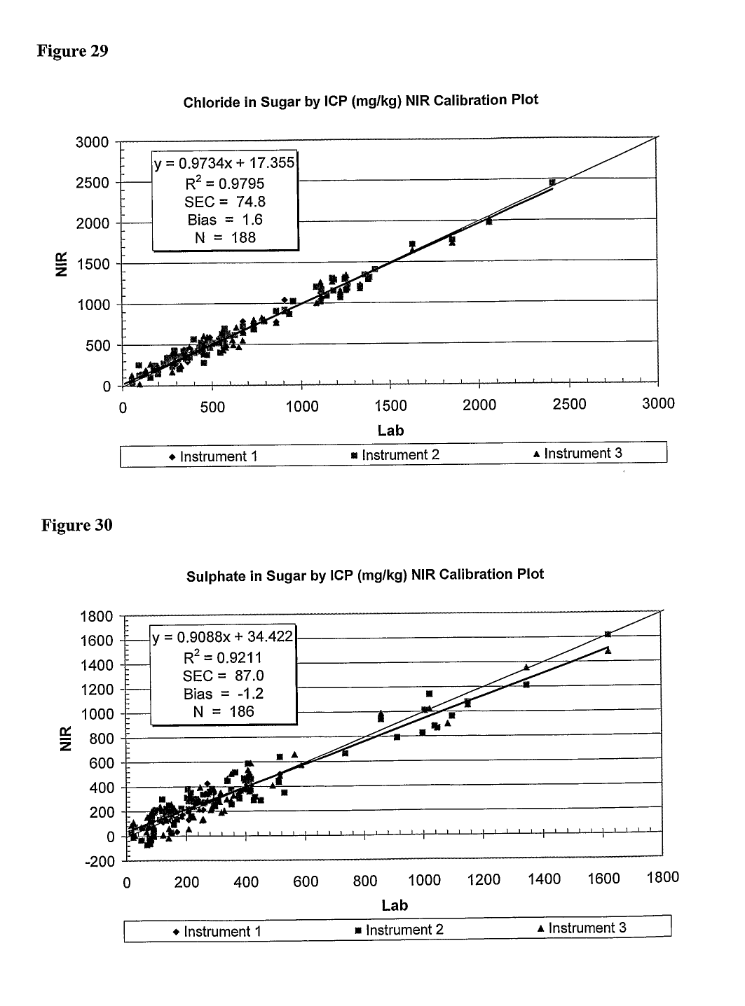

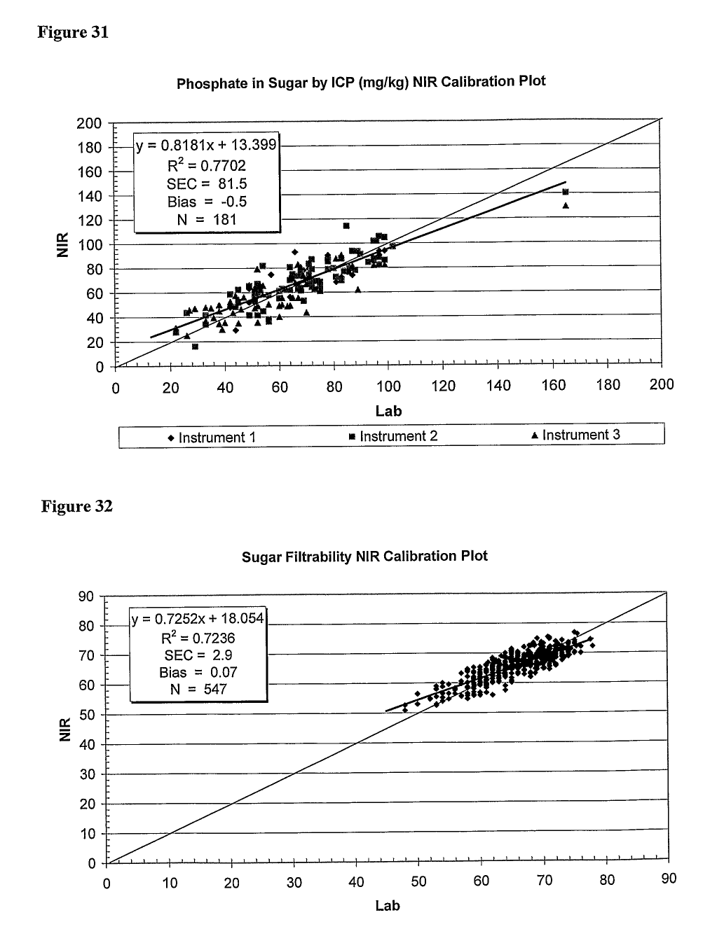

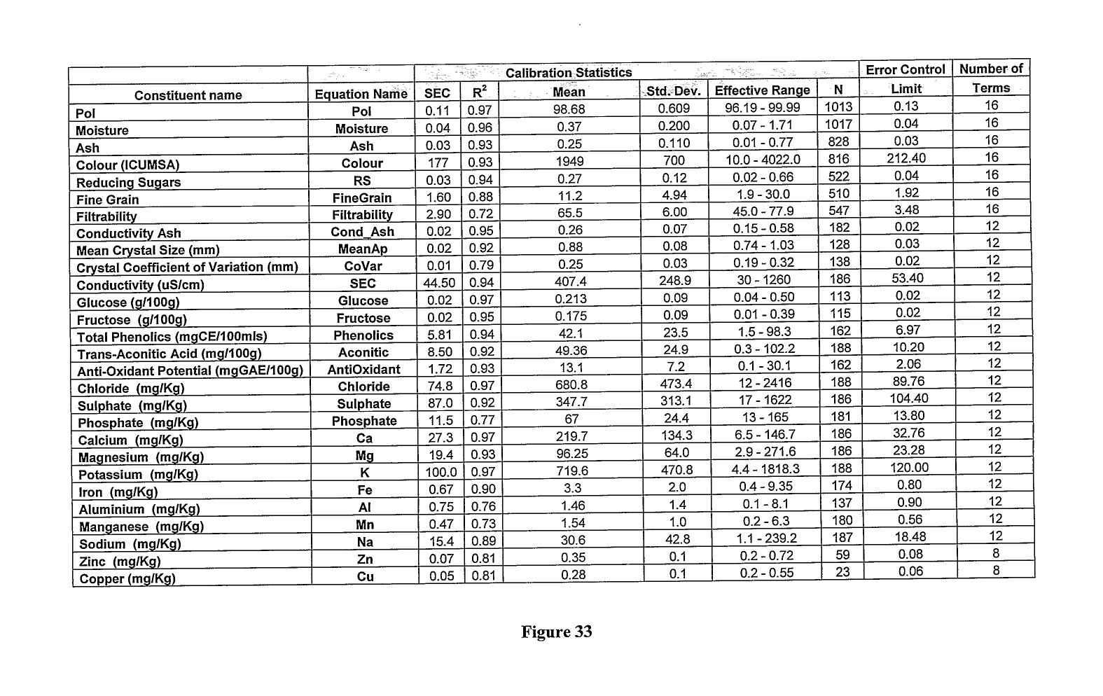

The results are illustrated in FIGS. 8 to 33. It is clear from the figures that the sample points are all close to the linear plot.

### Conclusion:

As the sample points reach linearity, the r² value moves closer to 1. FIGS. 8 to 34 in this example convincingly demonstrate a statistically significant correlation can be developed from substrate NIR spectra to derive concentrations of minerals, carbohydrates, organic acids and polyphenols. This method is therefore useful for a rapid on-line and/or offline measuring tool for QA/QC purposes in making sugar containing higher polyphenol levels. The method can be readily adapted and integrated into current industrial methodologies and process control systems.

The word ‘comprising’ and forms of the word ‘comprising’ as used in this description and in the claims does not limit the invention claimed to exclude any variants or additions. Modifications and improvements to the invention will be readily apparent to those skilled in the art. Such modifications and improvements are intended to be within the scope of this invention.

1. A process for the manufacture of sugar products having desired levels of specific phytochemicals comprising:
(a) preparing a primary sugar product;
(b) analysing the primary sugar product from step (a) for its phytochemical profile using an analytical method selected from the group consisting of near infrared spectroscopy, electrical conductivity and combinations thereof;
(c) comparing the phytochemical profile from step (b) with a reference phytochemical profile; and
at least one of:
(di) treating the primary sugar product, if required, to achieve a final sugar product having desired levels of specific phytochemicals;
or
(dii) altering the preparation process in step (a), if required, to produce a primary sugar product having the desired levels of specific phytochemicals.

2. The process according to claim 1, wherein the analysis in step (b) comprises:
(i) mounting a scanning head adjacent the extract (off-line) or processing stream (in-line), the scanning head comprising a remote light source and reflected light gathering and transmission apparatus;
(ii) using a monochromator of a near infrared spectrophotometer to resolve the reflected light into light of a discrete wavelength;
(iii) accessing a database containing a reference calibration equation linking absorption characteristics by discrete wavelengths with the quantified presence of each of the species of interest;
(iv) using a computer to create a profile of each of the species of interest by application of the calibration equation to the obtained spectrum for the extract or processing stream, and
(v) comparing the profile created with desired profile parameters stored in the database to identify the desired profile of the one or more species.

3. The process according to claim 1, wherein the primary sugar product is selected from the group consisting of sugar, extracts from sugar cane, extracts from sugar processing or waste streams, and mixtures thereof.

4. The process according to claim 1, wherein step (a) comprises the addition of affination syrup or a molasses extract to increase the levels of the desired phytochemical species.

5. The process according to claim 1, wherein step (b) comprises analysis of phytochemicals selected from the group consisting of polyphenols, antioxidants, organic acids, colorants, polysaccharides, soluble fibre, insoluble fibre, minerals, reducing sugars, policosanols, phytosterols, neutral lipids, phospholipids, emulsifiers, proteins and mixtures thereof.

6. The process according to claim 1, wherein the treatment in step (di) comprises spraying onto the primary sugar product a sugar cane extract.

7. The process according to claim 1, wherein the final sugar product is a low GI sugar.

8. A process for the manufacture of a food product comprising
(a) preparing a base phytochemical carrier;
(b) analysing the base phytochemical carrier using near infrared spectroscopy;
(c) comparing the profile from step (b) with a reference profile; and
at least one of
(di) treating the base phytochemical carrier, if required, to achieve a food product having desired levels of phytochemicals;
or
(dii) altering the preparation process in step (a), if required, to produce the base phytochemical carrier having the desired levels of phytochemicals.

9. The process according to claim 8, wherein the base phytochemical carrier is selected from the group consisting of soluble fibre, insoluble fibre, flour, cereals, dairy products and mixtures thereof.

10. The process according to claim 8, wherein steps (di) and (dii) comprise treating the base phytochemical carrier with an extract of a phytochemical source selected from the group consisting of cocoa beans and cocoa processing by-products, tea and tea processing waste streams, cocoa pod husks and shells, coffee beans, coffee waste, grape pomice, cereals, legumes, nuts, oilseeds, fruits, vegetables, beverages, herbal products, and mixtures thereof.

11. A process for the manufacture of an extract of sugar cane comprising:
(a) preparing a first extract of sugar cane;
(b) analysing the first extract of sugar cane from step (a) for its phytochemical profile using an analytical method selected from the group consisting of near infrared spectroscopy, electrical conductivity and combinations thereof;
(c) comparing the value from step (b) with a reference profile; and
at least one of:
(di) treating the first extract of sugar cane with further extraction processes, if required, to achieve the desired levels of phytochemicals;
or
(dii) altering the preparation process in step (a), if required, to produce an extract of sugar cane having the desired levels of phytochemicals.

12. A process for the manufacture of a low GI sugar product comprising:
(a) preparing a primary sugar product selected from the group consisting of standard crystalline raw sugar, mill white sugar and mixtures thereof;
(b) analysing the primary sugar product from step (a) using near infrared spectroscopy for its phytochemical profile;
(c) comparing the profile from step (b) with a reference phytochemical profile; and
(d) treating the primary sugar product, if required, by spray coating it with a molasses extract to achieve a final sugar product having the desired levels of specific phytochemicals.
